(19) United States
(12) Plant Patent Application Publication
(10) Pub. No.: US 2022/0071074 P1
(43) Pub. Date: Mar. 3, 2022

(54) Clematis plant named 'Evipo097'

(50) ;

(71) Applicants: Mogens Nyegaard Nyegaard Olesen, Fredensborg (DK); Raymond J. Evison, Vale (GB)

(72) Inventors: Mogens Nyegaard Nyegaard Olesen, Fredensborg (DK); Raymond J. Evison, Vale (GB)

(21) Appl. No.: 16/873,993

(22) Filed: Sep. 3, 2020

(51) Int. Cl. A01H 6/72

(52) U.S. Cl. PLT/228

(57) ABSTRACT

A new Clematis plant with a compact growth habit, profuse, white flowers, and continuous summer flowering. The variety successfully propagates from softwood cuttings and is suitable for cultivation in commercial nursery culture. This new and distinct variety has shown to be uniform and stable in the resulting generations from asexual propagation from vegetative cuttings.

Botanical classification: Genus: Clematis.

Species: viticella.

Variety denomination: ‘Evipo097’.

### SUMMARY OF THE CLAIMED PLANT

The present invention constitutes a new and distinct variety of Clematis plant which originated from a controlled crossing between the female seed parent, an un-named seedling, and the male pollen parent, an un-named seedling. Both parent varieties are non-patented.

The two parents were crossed during the summer of 2000 and the resulting seeds were planted the following winter in a controlled environment in Guernsey, Channel Islands, United Kingdom. The new variety named ‘Evipo097’ originated as a single seedling from the stated cross.

The new Clematis plant may be distinguished from its female seed parent and male pollen parent by the following characteristics. The female seed parent has mauve tepals while the new variety has white tepals. The male seed parent has mauve tepals while the new variety has white tepals.

The objective of the hybridization of this Clematis plant was to create a new and distinct variety for nursery culture with unique qualities such as:
1. Uniform and abundant white flowers;
2. Vigorous and compact growth, making the variety suitable for container culture; and
3. Improved disease resistance.

This combination of qualities was lacking in Clematis plants that were in commercial cultivation and the qualities have been substantially achieved in the new variety.

‘Evipo097’ was selected by Mogens N. Olesen and Raymond J. Evison in their Clematis development program in Guernsey, Channel Islands, United Kingdom in 2001. Asexual reproduction of ‘Evipo097’ by means of vegetative cuttings and traditional layering was first performed by Mogens N. Olesen and Raymond J. Evison in the nursery during the summer of 2001. This initial and subsequent asexual propagations have demonstrated that the characteristics of ‘Evipo097’ are true to type and are transmitted from one generation to the next.

### BRIEF DESCRIPTION OF THE DRAWING

The accompanying color illustrations show as true as is reasonably possible to obtain in color photographs of this type the typical characteristics of the buds, flowers, leaves, and stems, of ‘Evipo097’.

Specifically illustrated in FIG. 1 of the drawings are open flowers at various stages of development and flower buds.

FIG. 2 shows a flowering branch, bare stems, and leaves.

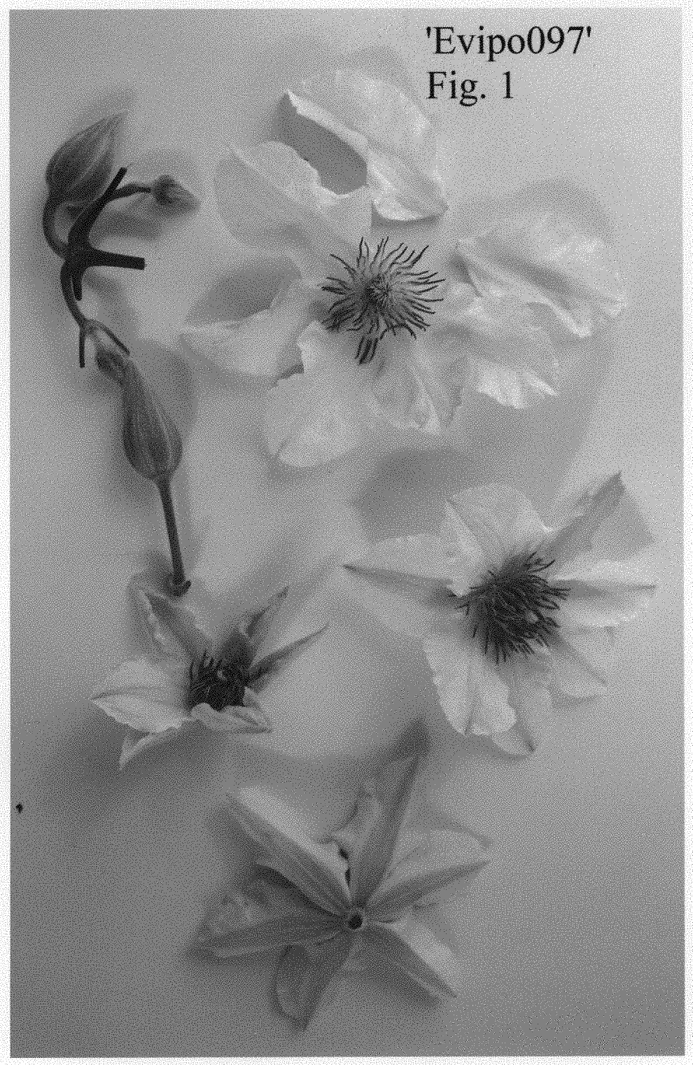

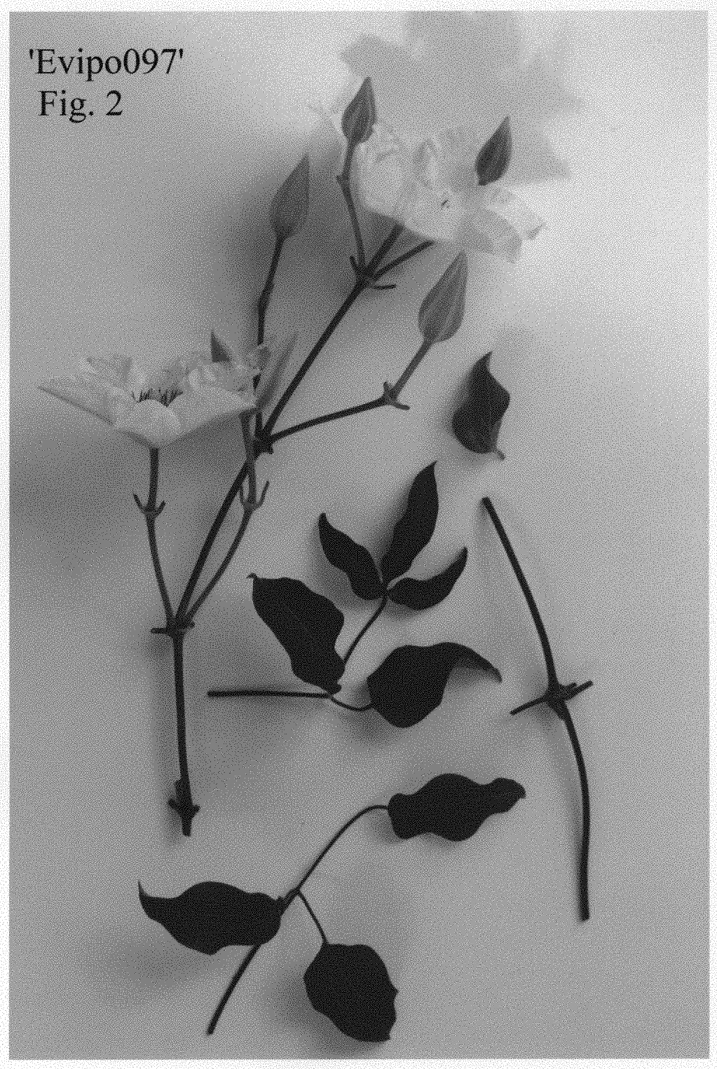

Illustrated plants are 2 years of age.

### DETAILED DESCRIPTION OF THE VARIETY

The following is a detailed description of ‘Evipo097’, as observed in its growth throughout the flowering period in Marion County Oreg. Observed plants were cultivated for a period of 24 months in 2 liter containers. Certain phenotypical characteristics of the variety may vary under different environmental, cultural, agronomic, seasonal, and climatic conditions. Color references are made using The Royal Horticultural Society (London, England) Colour Chart, 2001, except where common terms of color are used.

For a comparison, several physical characteristics of the Clematis variety ‘Evipo052’ described and illustrated in U.S. Plant Pat. No. 28,600 are compared to ‘Evipo097’ in Chart 1.

CHART 1

| | ‘Evipo097’ | ‘Evipo052’ |
|---|---|---|
| Flower diameter | 103 mm | 120 mm |
| Tepal upper | White Group 155C | White Group 155C |
| surface after | | |
| opening | | |
| Tepal count | 6 to 8 | 8 |

### FLOWER AND FLOWER BUD

Blooming habit: Repeat flowering. The natural flowering period is generally from April to July, then again September through October.
Flower bud:
   Size.—Normally 35 mm in length. Bud diameter is 13 mm.
   Bud form.—Long campanulate, broad based.
   Bud color.—Yellow-Green Group 145A and 145C.
   Texture.—Highly pubescent.
Pedicel:
   Surface texture.—Smooth.
   Length.—On average 22 mm in length with 4 mm diameter.
   Color.—Yellow-Green Group 145A.
   Strength.—Moderately strong.
Receptacle:
   Surface texture.—Highly pubescent.
   Shape.—Broad funnel.
   Size.—3 mm (h)×5 mm (w).
   Color.—Yellow-Green Group 145A.
Flower arrangement:
   Location on vine.—New growth only.
   Borne.—Singly, or in clusters of 2 to 3 flowers along axillary buds.
Flower bloom:
   Size.—On average, flowers are 103 mm in diameter and 22 mm in depth.
   Profile.—Open flowers are nearly flat.
   Fragrance.—None.
   Lasting quality.—Flowers normally remain up to 18 days on the plant.
Tepals:
   Tepal color.—Upon opening, the upper surface is Yellow-Green Group 145D. The lower surface is Green-White Group 157D with a central bar of Green White 145C. After opening, the upper surface is White Group 155C. The lower surface is White Group 155C with a central bar Green White 157A.
   Quantity.—Normally 6 to 8 tepals.
   Size.—53 mm in length by 34 mm wide.
   Shape.—Individual tepal shape is elliptic. The tepal apex is mucronate. The tepal base is obtuse.
   Apex recurvature.—Moderate.
   Tepal cross section.—Slightly cupped.
   Margins.—Entire. Moderate to strong undulations of margin.
   Persistence.—Tepals drop off cleanly.
Reproductive organs:
   Pollen.—Quantity: Moderate. Color: White Group 155A.
   Anthers.—Size: 6 mm in length. Color: Brown Group 200D. Quantity: On average, 55.
   Filaments.—Color: Green-White Group 157D. Length: 5 mm.
   Pistils.—Quantity: On average, 30.
   Stigmas.—Superior in location relative to the length of the filaments and the height of the anthers.
   Styles.—Color: Green-White Group 157A. Length: 10 mm.
Seed head characteristics: Seed not observed to date.

### PLANT

Plant form: Climbing.
Plant growth: Moderately vigorous.
Size: Seasons growth attains 80 to 90 cm in height. Average spread is 40 cm.
Stems:
   Color.—Juvenile stems are Yellow-Green Group 145A. Mature stems are Greyed-Orange Group 176A.
   Internodes.—On average, 65 cm between nodes.
   Length.—Normally 15 cm from the base of the plant to the flowering portion of the stem.
   Diameter.—About 3 mm.
   Texture.—Mature stems are ribbed and pubescent.
Plant foliage:
   Leaf characteristics.—Deciduous.
   Arrangement.—Trifoliate.
   Leaf size.—Compound leaves are about 155 mm (l)×115 mm (w). Leaflets are about 54 mm (l)×30 mm (w).
   Leaf color.—Juvenile upper Yellow-Green Group N144A. Juvenile lower Yellow-Green Group N144A. Mature upper Yellow-Green Group 146A. Mature lower Yellow-Green Group 146B.
   Stipules.—Absent.
   Petioles.—Size: Normally 45 mm in length by 1.5 mm diameter. Texture: Smooth. Color: Yellow-Green Group 144A and Greyed-Purple Group 183A.
   Petioloule.—Size: About 20 to 30 mm in length by 1.5 mm diameter. Texture: Pubescent. Color: Greyed-Purple Group 183A.
   Leaflet shape.—Elliptic. The base is rounded, apex acute.
   Margin.—Entire. Occasionally there are one or two deep clefts in the margin.
   Surface.—The upper side is smooth, the lower side is smooth.
   Thickness.—Not glossy.
Disease resistance: Subject to any disease that normally attacks the species. However the variety is more tolerant to Clematis wilt, Ascochyta clematidina, than some Clematis known to the inventors.
Cold hardiness: The variety is tolerant to USDA Hardiness Zone 6.
Heat tolerance: The variety has been found to be suitable for climate conditions found in the American Horticulture Society heat zone 7.

We claim:

1. A new and distinct variety of Clematis plant named ‘Evipo097’, substantially as described and illustrated , due to its abundant white flowers with good keepability, attractive long lasting foliage and compact growth, year round flowering under glasshouse conditions, suitability for production from softwood cuttings in pots, durable flowers and foliage which make the variety suitable for distribution in the floral industry.
